(19) United States
(12) Patent Application Publication
(10) Pub. No.: US 2022/0236292 A1
(43) Pub. Date: Jul. 28, 2022

(54) DETECTION OF DEBRIS IN RECIRCULATING PHAGOCYTES

(71) Applicant: ZELOSDX, INC., Tucson, AZ (US)

(72) Inventors: Uwe R. Muller, Gallatin, TN (US); Vanessa White, Tucson, AZ (US)

(21) Appl. No.: 17/719,243

(22) Filed: Apr. 12, 2022

Related U.S. Application Data
(63) Continuation-in-part of application No. 17/228,416, filed on Apr. 12, 2021.
Application No. 17/228,416 is a continuation-in-part of application No. 16/872,064, filed on May 11, 2020, now abandoned.
(60) Provisional application No. 62/845,670, filed on May 9, 2019.

(51) Int. Cl. G01N 33/68; G01N 33/569; G01N 33/58

(52) U.S. Cl. CPC G01N 33/6896; G01N 33/582; G01N 33/56972

(57) ABSTRACT

Methods for detecting neural-derived debris inside or on the cell surface of circulating neutrophils. The methods include single cell analysis of neutrophils and associated neural-derived biomarkers, using assays such as flow cytometry and single cell ELISA. The present invention also features methods for detecting and/or monitoring various states of brain health based on the neural-derived debris in said neutrophils.

### CROSS-REFERENCES TO RELATED APPLICATIONS

This application is a continuation-in-part of and claims priority to U.S. patent application Ser. No. 17/228,416 filed on Apr. 12, 2021, the specification of which is incorporated herein in its entirety by reference.

This application is also a continuation-in-part of and claims priority to U.S. patent application Ser. No. 16/872,064 filed May 11, 2020, which is a non-provisional and claims benefit of U.S. Provisional Application No. 62/845,670, filed May 9, 2019, the specification(s) of which is/are incorporated herein in their entirety by reference.

### BACKGROUND OF THE INVENTION

### Field of the Invention

The present invention relates to detection of compounds (e.g., proteins and/or other molecules) derived from neural tissue inside or displayed on the cell surface of recirculating phagocytes, which may be useful for detecting or monitoring or predicting risk for particular disease states or processing, including but not limited to disease states or processes related to brain health, such as a particular neurodegenerative disease, the presence of brain tissue damages, etc. The present invention includes preparation of compounds (e.g., proteins and/or other molecules) derived from neural tissue, wherein the compounds are inside or displayed on the cell surface of recirculating phagocytes. The present invention may include whole sample analysis, single-cell analysis, etc.

### Background Art

Inventors surprisingly discovered that neural tissue-derived debris (e.g., debris from the brain) can be detected (e.g., visualized, quantified, etc.) inside or on the cell surface of circulating neutrophils (e.g., instead of blood sample plasma). This was surprising since previous methods for detecting neural tissue-derived debris used PBMC preparations, which excluded neutrophils. Further, the use of neutrophils in the methods herein is not obvious since the phagocytic response of neutrophils has been primarily associated with pathogens and secretion of inflammatory mediators. The presence, and/or amount of, and/or ratio and/or pattern of debris may be useful for detecting and/or monitoring various disease states or processes (such as but not limited to neurological disease conditions or brain tissue damage), or for determining a risk for particular disease states or processes. Note the present invention is not limited to biomarkers associated with neurological disease or conditions.

### BRIEF SUMMARY OF THE INVENTION

The present invention describes methods for detecting neural-derived debris inside or on the cell surface of circulating neutrophils, and methods for detecting and/or monitoring various states of brain health based on the neural-derived debris in said neutrophils.

Analysis of neural-derived debris inside and/or on the cell surface of neutrophils in a sample may be achieved using a variety of methods. For example, samples (from a patient) may be subjected to an assay that allows for single cell analysis, e.g., analysis of individual neutrophils. In some embodiments, whole blood is used. In some embodiments, a sample preparation is used such as but not limited to cell isolates, e.g., isolated neutrophils. The assays may feature specific labeling and detection of neutrophils and the biomarkers associated with said neutrophils (e.g., inside and/or on the cell surface). Methods include but are not limited to flow cytometry based assays, single cell ELISA, microscopy-based assays, etc. As an example, in some embodiments, the neutrophils are immobilized and stained for one or more brain-derived biomarkers (e.g., a biomarker that would only normally be found in brain or CNS tissue). The present invention is not limited to single cell analysis. For example, in some embodiments, neutrophils are isolated and then a lysate prepared for an assay to detect the neural-derived biomarkers.

For any of the embodiments herein, the neutrophils may be obtained from peripheral blood, synovial fluid, cerebrospinal fluid (CSF), or other sources as described herein. For example, in certain embodiments, the phagocytes are obtained from blood, e.g., drops of blood obtained via venipuncture or a finger prick. The present invention is not limited to the aforementioned samples or methods for obtaining samples.

The presence, and/or amount of, and/or ratio and/or pattern of debris may be indicative of various disease states or processes (such as but not limited to neurological disease conditions or brain tissue damage), or indicate risks for particular disease states or processes. Note the present invention is not limited to biomarkers associated with neurological disease or conditions.

Without wishing to limit the present invention to any theory or mechanism, the present invention may provide close to real-time data on what is happening in the brain since that particular cargo may only be present inside or on the cell surface of the recirculating phagocytes for a certain length of time (e.g., a few days) before it is completely digested.

The present invention is not limited to humans. As used herein, a patient or subject may refer to an animal such as but not limited to a mammal. Mammals may include but are not limited to primates (e.g., a human, non-human primates), a mouse, a rat, a llama, a rabbit, a dog, a primate, a guinea pig, a cat, a hamster, a pig, a goat, a horse, or a cow. The present invention is not limited to the aforementioned subjects or patients.

The present invention also includes methods for preparation of cells and/or CNS-derived debris for analysis. For example, the present invention includes methods of preparing neutrophils containing CNS-derived (e.g., brain-derived, neural-derived) compounds (e.g., the debris or biomarkers that would only normally be found in CNS tissue such as but not limited to brain) for analysis. The present invention also features preparation of the CNS-derived compounds found in the circulating neutrophils. The present invention also features preparation of blood samples for analyzing the CNS-derived compounds found in the circulating neutrophils. The present invention is not limited to isolation of circulating neutrophils and creating a lysate. The present invention also includes methods using whole blood. The present invention also includes methods for single-cell analysis. Non-limiting examples of methods described herein include flow cytometry, ELISA, FACS, and fluorescent staining. In some embodiments, the analysis is single cell analysis, e.g., using flow cytometry, single cell ELISA, etc. Such techniques are known in the art. For example, in single cell ELISA, cells are captured and immobilized. In some cases, the cells are permeabilized and antibodies are added directed to biomarkers and subsequently subjected to imaging (e.g., via microscopy, direct imaging, etc.). In some cases, the cells are immobilized and lysed, and biomarkers are captured via specific antibodies that have also been immobilized on the same surface. After washing to remove unbound materials, a secondary labeled antibody specific for the biomarker(s) is added, which after washing is then analyzed by imaging. The present invention includes the use of systems (e.g., microfluidic devices, etc.) that allow single cell trapping and analysis by ELISA. Details can be found in Yin and Marshall (2012, Cur. Op. Biotechnology 23:110-119), Spiller et al. (2010, Nature 465:736-745), among others. The present invention includes the use of systems (e.g., microfluidic devices, etc.) that allow single cell trapping and analysis by ELISA. The present invention is not limited to these particular methods or systems for single cell analysis.

The methods herein for preparing central nervous system (CNS)-derived (e.g., brain-derived) compounds may comprise introducing to a whole blood sample obtained from outside central nervous system (CNS) tissue of a subject a first detectable binding moiety specific for circulating neutrophils and a second detectable binding moiety specific for a CNS-derived molecule, the first detectable binding moiety being differentially detectable from the second detectable binding moiety; subjecting the preparation to single-cell analysis for detecting the first detectable binding moiety and second detectable binding moiety; and analyzing the CNS-derived molecules in the preparation.

In some embodiments, the method comprises producing a preparation comprising CNS-derived molecules by introducing to a whole blood sample obtained from outside central nervous system (CNS) tissue of a subject a first detectable binding moiety specific for circulating neutrophils and a second detectable binding moiety specific for a CNS-derived molecule, the first detectable binding moiety being differentially detectable from the second detectable binding moiety; and analyzing the CNS-derived molecules in the preparation.

The methods herein for preparing and/or analyzing central nervous system (CNS)-derived (e.g., brain-derived) compounds may comprise single-cell analysis of circulating neutrophils from a fluid sample obtained from outside central nervous system (CNS) tissue of a subject; and analysis of the CNS-derived compounds in the cells.

The present invention also provides methods for preparing and/or analyzing CNS-derived compounds wherein the CNS-derived compound is displayed on the cell surface of the neutrophils. For example, the method may comprise extracting circulating neutrophils from a fluid sample obtained from outside central nervous system (CNS) tissue of a subject; and producing a fraction of the extracted circulating neutrophils by separating neutrophils with membrane-bound CNS-derived peptides/compounds from neutrophils without membrane-bound CNS-derived peptides/compounds. The fraction of the neutrophils may comprise the neutrophils with membrane-bound CNS-derived peptides/compounds. In some embodiments, the method further comprises analyzing the neutrophils in the fraction. In some embodiments, the method comprises lysing the whole sample as described herein, rather than first extracting the circulating phagocytes. In some embodiments, the method comprises single-cell analysis as described herein.

The present invention also includes the use of nanoparticles. Nanoparticles may be used to determine the presence and/or amount of a particular biomarker (e.g., epitope) in a particular cell or group of cells. In some embodiments, the nanoparticles are noble metal nanoparticles or alloys of noble metals. In some embodiments, the nanoparticles are gold nanoparticles, silver nanoparticles, or a combination thereof. In some embodiments, the nanoparticles are rods, spheres, or a combination thereof. In some embodiments, the nanoparticles have a diameter of 2 nm to 250 nm. In some embodiments, the biophysical properties refer to the adsorption or emission of electromagnetic waves by the nanoparticles in response to incident electromagnetic waves. In some embodiments, the biophysical properties refer to surface plasmon resonance. In some embodiments, the differential biophysical properties are measured by dynamic light scattering or tunable resistive pulse sensing.

The present invention also includes methods for measuring a biomarker inside of or displayed on a cell surface of neutrophils in a sample from a subject. In certain embodiments, the method comprises labeling neutrophils with a neutrophil-specific binding moiety; the method may further comprise staining at least one target biomarker molecule with a labeled target biomarker-specific binding moiety. The target biomarker is or comprises a compound derived from central nervous system tissue that is within or displayed on the cell surface of the phagocytes. The method may further comprise analyzing the binding moieties, e.g., measuring a ratio, measuring an amount, etc.

In some embodiments, the binding moiety is a fluorescently labeled binding moiety. In some embodiments, the neutrophils are immobilized on a solid surface prior to staining. Solid surfaces may include but are not limited to microscope slides. In some embodiments, the solid surface is coated with one or more phagocyte-specific binding moieties, and neutrophils are immobilized on the solid surface through binding to these moieties. In some embodiments, the neutrophils are immobilized on a solid surface directly from blood, and other cells and blood components are removed from said surface through washing of the surface.

In some embodiments, the binding moieties are monoclonal or polyclonal antibodies, single-chain antibodies, single-chain proteins, binding peptides, or aptamers.

In some embodiments, biomarkers or biomarker fragments are detected inside or displayed on the surface of the neutrophils, and detection is possible via attachment of labeled fragment-specific binding moieties.

For any of the embodiments herein, the methods may be used to detect one biomarker. In some embodiments, the methods are used to detect two or more target biomarkers simultaneously. In some embodiments, the method detects two or more epitopes located on one or more target biomarker molecules. In some embodiments, the method comprises applying three or more binding moieties, each specific for a different epitope located on one or more target biomarker molecules. In some embodiments, two or more binding moieties are used to detect two or more structural conformations of the target biomarker. In some embodiments, a binding moiety used to detect the target biomarker is specific for a particular structural conformation of the target biomarker. In some embodiments, the structural conformation is a result of degradation or digestion of the target biomarker. In some embodiments, the structural conformation is a result of degradation or digestion of the target biomarker. In some embodiments, multiple different biomarkers are detected simultaneously by use of differentially labeled biomarker-specific binding moieties.

In some embodiments, the CNS-derived molecule is GFAP. In some embodiments, the CNS-derived molecule is Tau. In some embodiments, the CNS-derived molecule is GFAP, Tau, or both. In some embodiments, the CNS-derived molecule comprises one or a combination of: Tau, phosphorylated Tau, hippocalcin-1, 14-3-3 protein, MBP, UCH-L1, TDP-43, superoxide dismutase (SOD), neuromelanin, glial fibrillary acidic protein (GFAP), neurofilament light chain (NFL), neurofilament heavy chain (NFH), neurofilament medium chain (NFM), phosphorylated NFL, phosphorylated NFH, phosphorylated NFM, internexin (Int), peripherin, UCH-L1, amyloid beta, alpha-synuclein, apo A-I, Apo E, Apo J, a viral antigen, a JC viral antigen, TGF-beta, VEGF, dopamine-beta-hydroxylase (DBH), vitamin D binding protein, histidine-rich glycoprotein, cDNA FLJ78071, apolipoprotein C-II, immunoglobulin heavy constant gamma 3, alpha-1-acid glycoprotein 1, alpha-1-acid glycoprotein 2, haptoglobin-related protein, leucine-rich alpha-2-glycoprotein, erythropoietin (EPO), C-reactive protein, tyrosinase EC 1.14.18.1, tyrosine hydroxylase, tyrosinase EC 1.14.16.2, PSD-95 protein, neurogranin, SNAP-25, TDP-43, transketolase, NSI associated protein 1, major vault protein, synaptojanin, enolase, alpha synuclein, S-100 protein, Neu-N, 26S proteasome subunit 9, ubiquitin activating enzyme ZE1, ubiquitin B precursor, vimentin, 13-3-3 protein, NOGO-A, neuronal-specific protein gene product 9.5, proteolipid protein; myelin oligodendrocyte glycoprotein, neuroglobin, valosin-containing protein, brain hexokinase, nestin, synaptotagmin, myelin associated glycoprotein, myelin basic protein, myelin oligodendrocyte glycoprotein, myelin proteolipid protein, annexin A2, annexin A3, annexin A5, annexin A6, annexin A11, ubiquitin activating enzyme ZE1, ubiquitin B precursor, vimentin, glyceraldehyde-3-phosphate dehydrogenase, 14-4-4 protein, rhodopsin, all-spectrin breakdown products (SBDPs), or a breakdown product thereof.

The present invention is not limited to the aforementioned biomarkers or antigens. In some embodiments, the biomarker may be a partially digested version of the aforementioned biomarkers. In some embodiments, the target biomarker molecule is associated with neurologic disease or neurotrauma. The biomarker may be selected based on its association with a particular disease or condition.

In some embodiments, the level of the biomarker detected inside of or displayed on the cell surface of the neutrophils is compared to a predetermined threshold to determine if it is abnormal. The predetermined threshold may be an industry standard. In some embodiments, the predetermined threshold is a laboratory standard. In some embodiments, the predetermined threshold is a patient standard. For example, in certain embodiments, the predetermined threshold is a level of the biomarker (or biomarkers) in neutrophils isolated from a fluid sample obtained from the patient at a particular time point, e.g., before administration of one or more therapeutic compositions. Each particular biomarker of interest may have its own predetermined threshold. With respect to predetermined thresholds and controls, the threshold or control may be a level of the particular biomarker or panel of biomarkers in non-diseased control samples. In some embodiments, the predetermined threshold or control is a level of the particular biomarker or panel of biomarkers at a given time point. The present invention is not limited to the aforementioned examples of thresholds or controls.

In some embodiments, central nervous system tissue damage, central nervous system repair, neurodegeneration or inflammation is associated with Multiple Sclerosis, Alzheimer's disease, mild cognitive impairment, Parkinson's disease, Multiple System Atrophy, Lewy body Disease, Progressive Supranuclear Atrophy, Corticobasal Degeneration, Amyotrophic Lateral Sclerosis, Huntington's Disease, concussion, Traumatic Brain Injury, Chronic Traumatic Encephalopathy, Macrocephaly, Hydrocephalus, Cerebral Hypoxia, REM sleep behavior disorder, or a disease causing secondary central nervous system damage. In some embodiments, the central nervous system tissue damage, central nervous system repair, or neurodegeneration causes cognitive impairment, motor disturbances, or both.

The present invention also features methods for creating patient cohorts (e.g., of one or more patients) for a clinical trial, e.g., a clinical trial for testing a therapeutic composition or therapeutic intervention for an effect on central nervous system (CNS) tissue damage, CNS repair, or neurodegeneration, etc. The CNS tissue damage, CNS repair, neurodegeneration, etc., may be associated with a particular disease, condition, trauma, etc., as described herein. The method features detecting a biomarker or antigen from, or produced by, the central nervous system (e.g., a brain antigen, etc.) utilizing the methods described herein.

In some embodiments, the method comprises: selecting at least one patient with active CNS disease or trauma by detecting a level of a biomarker inside of or displayed on the cell surface of the phagocytes from the fluid sample of the patient, the biomarker being associated with CNS tissue damage, CNS system repair, or neurodegeneration, etc., using the methods described herein. The fluid sample is from outside of a central nervous system tissue (e.g., outside of the brain tissue) of the patient. In some embodiments, if the level of the biomarker inside of or displayed on the cell surface of the phagocytes is abnormal, then the patient has active CNS disease or trauma.

The method may comprise: administering to the at least one patient with active CNS disease or trauma the therapeutic composition or therapeutic intervention; and detecting a level of a biomarker inside of or displayed on the cell surface of the phagocytes from the fluid sample of the patient, the biomarker being associated with CNS tissue damage, CNS system repair, or neurodegeneration, etc., using the methods described herein. The fluid sample is from outside of a central nervous system tissue (e.g., outside of the brain tissue) of the patient, e.g., after a predetermined amount of time following administration of the therapeutic composition or therapeutic intervention. In some embodiments, (a) if the level of the biomarker inside of or displayed on the cell surface of the phagocytes from a patient is normal, then the therapeutic composition or therapeutic intervention has a positive effect on CNS tissue damage, CNS repair, or neurodegeneration; (b) if the level of the biomarker inside of or displayed on the cell surface of the phagocytes from a patient is within a predetermined threshold associated with improved CNS disease or trauma, then the therapeutic composition or therapeutic intervention has a positive effect on CNS tissue damage, CNS repair, or neurodegeneration; or (c) if the level of the biomarker inside of or displayed on the cell surface of the phagocytes from a patient is improved compared to a level of the biomarker in phagocytes in a sample obtained from the patient at a time point prior to administration of the therapeutic composition or therapeutic intervention, then the therapeutic composition or therapeutic intervention has a positive effect on CNS tissue damage, CNS repair, or neurodegeneration.

As previously discussed, the predetermined threshold may be an industry standard, a laboratory standard, a patient standard, etc. Each particular biomarker of interest may have its own predetermined threshold. In some embodiments, a level of biomarker above the predetermined threshold is an abnormal level of the biomarker. In some embodiments, a level of biomarker below the predetermined threshold is an abnormal level of the biomarker. With respect to predetermined thresholds and controls, the threshold or control may be a level of the particular biomarker or panel of biomarkers in non-diseased control samples. In some embodiments, the predetermined threshold or control is a level of the particular biomarker or panel of biomarkers at a given time point. The present invention is not limited to the aforementioned examples of thresholds or controls.

For any of the embodiments herein, the therapeutic composition or therapeutic intervention may include but is not limited to: Mastinab, Daclizumab, Zinbryta, ER-Beta agonist, cyclophosphamide, rHIgM22, ponesimod, alpha-4 Integrin, AMP-110, an antisense oligonucleotide, Immune tolerizing agent, MultiStem, Kv1.3 Blocker, Alemtuzumab, IFNb-1 b, BHT-3009-01, IFNb-1a, dimethyl fumarate, IFNb-1a (PEGylated), Natalizumab, MT1301, Abatacept, RCP1063, a compound related to Iamitrigene, CNM-Au8, Mesenchymal stem cell transplant, apolipoprotein E-based, modified peptidomimetic, Aimspro, Anti-BAFF Human Ab, NDC-1308, GNbAC1, Vatelizumab, Ofatumumab, ozanezumab (Nono-A mAb), Firategrast (SB-683699, T-0047), Interferon Alfa N3, Mitoxantrone, Ibudilast, Simvastatin, Raltegravir, Plovamer Acetate, Vedolizumab, Siponimod, Secukinumab, Fingolimod, Copaxone, LSD1-MAOB, Amiloride, adrenocorticotropic hormone, Rituximab, Ocrelizumab, Teriflunomide, glatiramer acetate, Laquinimod, TG-1101 (ublituximab), Clemastine Fumarate, Lipoic Acid, Minocycline, Riluzole, MIS416, Fibrin, ATX-MS1467, ARX424, Tcelna, TBC-4746, Feldetrex, PV-267, RNS60, R1295, VSN16R, Angiotensin AT2 agonist, GEH 120714, PDDIA, PDD3a, Zonisamide, deferiprone, NTCELL, Caffine, Isradipine, Rivastigmine, Inosine, Rasagiline, GM1 ganglioside, Nicotine, Exenatide, Pioglitazone, Glycerol Phenylbutyrate, EPI-743, an anti-inflammatory drug, ANX005, AADvac1, 5HT-6, Verubestat, PXT864, Donepezil, NPT088, RG1450, RG7412, PDE4E, PDE4B, Methylthioninium chloride, T3D-959, MultiTEP, ABT957, Anti-Tau antibodies, Aducanumab, Solanezumab, or Inbrija.

The present invention also features methods of selecting a candidate for monitoring mild cognitive impairment or Parkinson's disease. The method comprises detecting one or more biomarkers of interest using the methods described herein. The method may comprise selecting the patient for monitoring for mild cognitive impairment or Parkinson's disease based on the patient having active central nervous system disease.

In some embodiments, the candidate has REM sleep behavior disorder (RBD). The biomarker may include but is not limited to dopamine-beta-hydroxylase (DBH), vitamin D binding protein, histidine-rich glycoprotein, cDNA FLJ78071, apolipoprotein C-II, immunoglobulin heavy constant gamma 3, alpha-1-acid glycoprotein 1, alpha-1-acid glycoprotein 2, haptoglobin-related protein, leucine-rich alpha-2-glycoprotein, C-reactive protein, Tau, phosphorylated Tau, hippocalcin-1, or 14-3-3 protein, MBP, UCH-L1, TDP-43, superoxide dismutase (SOD), neuromelanin, GFAP, neurofilaments light chains (NFL), UCH-L1, amyloid beta, alpha-synuclein, apo A-I, Apo E, Apo J, JC viral antibody titers, TGF-beta, VEGF, the like, or a combination thereof. The present invention is not limited to the aforementioned biomarkers or antigens. The biomarker may be selected based on its association with a particular disease or condition.

In some embodiments, an autoimmune disease or condition associated with neurodegeneration may be multiple sclerosis. In some embodiments, the disease or condition causing cognitive impairment may be Alzheimer's disease, or Lewy body disease. In some embodiments, the disease or condition causing motor disturbances is Parkinson's disease, Multiple Sclerosis, Supranuclear Atrophy, or Corticobasal Degeneration.

The present invention also features preservation of samples for preserving the amount and/or structure and/or location of the biomarker(s) of interest (e.g., for preserving the amount and/or structure and/or location of the epitope(s) of interest). For example, the present invention provides methods for treating samples for the purposes of preserving the biomarker, e.g., via heat denaturation (wherein proteolytic enzymes or other factors are inhibited without affecting the biomarker, e.g., the epitope of the biomarker, to a large extent). Other methods of preservation may include freeze drying or other rapid freezing processes, application of heparin or other factors, modifying the pH of the sample, etc. The present invention is not limited to the aforementioned methods or compositions.

Without wishing to limit the present invention to any theory or mechanism, it is believed that biomarkers that are associated with particular disease states of interest (e.g., biomarkers found in the re-circulating phagocytes as described herein) will continue to be discovered. Since the methods herein are not necessarily limited by the particular biomarker but instead features the phagocytic shuttle wherein the phagocytes are shuttles for CNS-derived debris indicative of processes occurring in the CNS and steps for detecting the biomarkers inside or on the surface of the shuttle phagocytes, the present invention includes those biomarkers that will be discovered in the future.

The present invention is not limited to fluorescent assays, e.g., fluorescent microscopy or imaging. In some embodiments, the methods herein comprise colorimetric assays. As a non-limiting example, the methods may comprise a colorimetric ELISA. In some embodiments, the methods herein comprise imaging without a microscope. In some embodiments, the methods herein comprise using an image analysis system, which may provide images from surfaces such as a slide or a plate (e.g., microplate well), etc.

The methods herein may be used for methods of detecting biological changes in CNS tissue. The methods may be performed in lieu of obtaining imaging of the subject or obtaining a biopsy.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows the results of GFAP fluorescent imaging and cell counts. Three image planes (GFAP1, GFAP2, GFAP3) from one sample were imaged at 5× and fluorescent cells (DAPI[350]/CD14[568]/GFAP[488]) were counted using ImageJ software. Cell counts were fairly high (low thousands). This sample contained on average 10.8% CD14+ cells and of those cells roughly 1.7% were GFAP+ (0.19% of total). There were a few cells which were GFAP+/CD14−.

FIG. 2 shows the results of Tau fluorescent imaging and cell counts. Three image planes (Tau1, Tau2, Tau3) from one sample were imaged at 5× and fluorescent cells (DAPI[350]/CD14[568]/TAU[488]) were counted using ImageJ software. Cell counts were somewhat lower (due to division of sample between several slides for troubleshooting). This sample contained on average 8.4% CD14+ cells and of those cells roughly 8.7% were TAU+ (0.7% of total). There were a few cells which were TAU+/CD14−.

### DETAILED DESCRIPTION OF THE INVENTION

As previously discussed, the present invention describes methods for detecting neural-derived debris inside or on the cell surface of circulating neutrophils, and methods for detecting and/or monitoring various states of brain health based on the neural-derived debris in said neutrophils.

As a non-limiting example, the methods of the present invention may comprise producing a preparation comprising CNS-derived molecules by introducing to a whole blood sample obtained from outside central nervous system (CNS) tissue of a subject a first detectable binding moiety specific for circulating neutrophils and a second detectable binding moiety specific for a CNS-derived molecule, the first detectable binding moiety being differentially detectable from the second detectable binding moiety; subjecting the preparation to single-cell analysis for detecting the first detectable binding moiety and second detectable binding moiety; and analyzing the CNS-derived molecules in the preparation.

In some embodiments, the single-cell analysis is flow cytometry. In some embodiments, the single-cell analysis is a single cell enzyme linked immunosorbent assay (ELISA). In some embodiments, the single-cell analysis is a microscopy-based assay. In some embodiments, the single-cell analysis comprises placing the preparation on a solid surface, using said surface as a wave guide for illumination, and imaging by direct charge-coupled device (CCD). In some embodiments, the first detectable binding moiety, the second detectable binding moiety, or both comprise a fluorescent label, a fluorescent antibody, a nanoparticle, a quantum dot, or a tag.

The present invention also features methods for measuring a biomarker inside of or displayed on a cell surface of neutrophils in a sample from a subject. In some embodiments, the method comprises staining neutrophils in the sample from the subject with a labeled first binding moiety specific for neutrophils, and staining at least one target biomarker molecule with a labeled second binding moiety specific for the target biomarker, the first binding moiety can be differentiated from the second binding moiety, the target biomarker being a compound derived from central nervous system tissue that is within or displayed on the cell surface of the neutrophils; and detecting the first binding moiety and the second binding moiety, wherein the relationship between the amount or distribution of the first binding moiety and second binding moiety is indicative of an amount of target biomarker molecules inside or displayed on the cell surface of said neutrophils.

In some embodiments, the sample is whole blood. In some embodiments, detecting the binding moieties comprises subjecting the sample to flow cytometry or ELISA. In some embodiments, the neutrophils are immobilized prior to staining. In some embodiments, the labels of the binding moieties are fluorescent labels. In some embodiments, the target biomarker molecule is associated with neurologic disease or neurotrauma.

For any of the embodiments herein, the target biomarker may be one or a combination of: Tau, phosphorylated Tau, hippocalcin-1, 14-3-3 protein, MBP, UCH-L1, TDP-43, superoxide dismutase (SOD), neuromelanin, glial fibrillary acidic protein (GFAP), neurofilament light chain (NFL), neurofilament heavy chain (NFH), neurofilament medium chain (NFM), phosphorylated NFL, phosphorylated NFH, phosphorylated NFM, internexin (Int), peripherin, UCH-L1, amyloid beta, alpha-synuclein, apo A-I, Apo E, Apo J, a viral antigen, a JC viral antigen, TGF-beta, VEGF, dopamine-beta-hydroxylase (DBH), vitamin D binding protein, histidine-rich glycoprotein, cDNA FLJ78071, apolipoprotein C-II, immunoglobulin heavy constant gamma 3, alpha-1-acid glycoprotein 1, alpha-1-acid glycoprotein 2, haptoglobin-related protein, leucine-rich alpha-2-glycoprotein, erythropoietin (EPO), C-reactive protein, tyrosinase EC 1.14.18.1, tyrosine hydroxylase, tyrosinase EC 1.14.16.2, PSD-95 protein, neurogranin, SNAP-25, TDP-43, transketolase, NSI associated protein 1, major vault protein, synaptojanin, enolase, alpha synuclein, S-100 protein, Neu-N, 26S proteasome subunit 9, ubiquitin activating enzyme ZE1, ubiquitin B precursor, vimentin, 13-3-3 protein, NOGO-A, neuronal-specific protein gene product 9.5, proteolipid protein; myelin oligodendrocyte glycoprotein, neuroglobin, valosin-containing protein, brain hexokinase, nestin, synaptotagmin, myelin associated glycoprotein, myelin basic protein, myelin oligodendrocyte glycoprotein, myelin proteolipid protein, annexin A2, annexin A3, annexin A5, annexin A6, annexin A11, ubiquitin activating enzyme ZE1, ubiquitin B precursor, vimentin, glyceraldehyde-3-phosphate dehydrogenase, 14-4-4 protein, rhodopsin, all-spectrin breakdown products (SBDPs), or a breakdown product thereof.

The present invention is not limited to fluorescence microscopy or fluorescently labeled binding agents (e.g., antibodies). Any appropriate imaging system may be considered (e.g., colorimetric, etc.).

In some embodiments, the sample is a blood sample. The present invention is not limited to blood samples and may include other fluids such as but not limited to cerebrospinal fluid (CSF). A subject includes a mammal. A mammal may include but is not limited to a human, a mouse, a rat, a llama, a rabbit, a dog, a primate, a guinea pig, a cat, a hamster, a pig, a goat, a horse, or a cow. The present invention is not limited to the aforementioned subjects or patients.

In some embodiments, the blood sample is one or a few drops of blood (e.g., from a blood draw, a finger prick, etc.). For example, the blood sample may be 1-2 drops of blood, 1-4 drops of blood, 4-10 drops of blood, etc. The present invention is not limited to the aforementioned volumes of samples.

Without wishing to limit the present invention to any theory or mechanism, it is believed that the detection methods herein are advantageous. For example, the methods herein do not require a large amount of sample (e.g., a few drops of blood may be obtained from a finger prick).

The methods herein may further comprise introducing to the sample a molecule for inhibiting degradation (or further degradation) of the neural-derived compound (e.g., CNS-derived compound, CNS-derived debris, etc.) in or on the phagocytes. For example, generally, any component that increases the pH of the phagolysosomes, which would inhibit the enzymes in the phagolysosomes, may help reduce the degradation of peptides (e.g., the biomarkers of interest) in the phagolysosomes. In some embodiments, the molecule for inhibiting further degradation of the neural-derived biomarker in the phagolysosome of the phagocytes comprises one or a combination of phagolysosomal protease inhibitors. In some embodiments, the protease inhibitor comprises leupeptin. In some embodiments, the molecule for inhibiting further degradation of the neural-derived biomarker in the phagolysosome of the phagocytes comprises a molecule that increases the pH of the phagolysosomes of the phagocytes in the first fluid sample. In some embodiments, the molecule for increasing the pH of the phagolysosomes of the phagocytes in the first fluid sample comprises an alkaline buffer. Alkaline buffers are well known to one of ordinary skill in the art, e.g., chloroquin, carbonate/bicarbonate buffer, buffers of pH 9.2 or above, weak base buffers, quinine, etc. In some embodiments, both a phagolysosomal inhibitor and alkaline buffer are added. In some embodiments, a protease inhibitor is introduced to the sample within 1 minute, 2 minutes, 3 minutes, 4 minutes, or 5 minutes, or within 10 minutes, 15 minutes, 20 minutes, etc., of when the sample is obtained.

The phagocytes may be obtained, collected, concentrated, etc. via a variety of means. For example, methods may feature a cell-affinity chromatography system herein the phagocytes interact with and/or bind a ligand immobilized on a system such as a filter, a membrane, a slide, a column, etc. The phagocytes may then be eluted after being captured by the chromatography system. In certain embodiments, the ligand is an antibody that is specific for the cell type of interest, e.g. the phagocyte. As a non-limiting example, the chromatography system may feature a spin column with a resin displaying a phagocyte-specific antibody, wherein the sample (e.g., blood) is introduced to the spin column. In some embodiments, the system features a slide displaying a phagocyte-specific antibody, wherein the sample (e.g., blood) is introduced to the slide. In some embodiments, the system features a syringe with a membrane displaying a phagocyte-specific antibody, wherein the sample (e.g., blood) is introduced to the syringe. In some embodiments, the method comprises introducing magnetic beads to the sample, whereupon phagocytes engulf the magnetic beads, yielding magnetic phagocytes. The method may further comprise separating the magnetic phagocytes using a magnetic separation mechanism. In some embodiments, the method comprises introducing to the sample a stimulator to stimulate phagocytosis of the magnetic beads by the phagocytes. In some embodiments, the magnetic beads are conjugated with an acid hydrolase inhibitor. In some embodiments, the magnetic beads are conjugated with an antibody or antibody component to stimulate phagocytosis. In some embodiments, the magnetic beads are introduced to the first fluid sample within 1 minute, 2 minutes, 3 minutes, 4 minutes, 5 minutes, 10 minutes, 15 minutes, or 20 minutes of when the first fluid sample is obtained. In some embodiments, the magnetic beads/particles are coated with a phagolysosomal inhibitor (e.g., leupeptin). In some embodiments, the magnetic beads/particles are coated with a mix of compounds, e.g., a phagolysosomal inhibitor (e.g., leupeptin), an antibody (e.g., IgG, IgG(Fc), etc.).

In some embodiments, the magnetic separation mechanism comprises a magnetic column or magnetic rack. In some embodiments, the container (for the blood sample) comprises Ficoll. In some embodiments, the container (for the blood sample) does not comprise Ficoll or is free of Ficoll. In some embodiments, the magnetic phagocytes are separated using the magnetic separation mechanism within 1 hour of harvesting of the first fluid sample. In some embodiments, the magnetic phagocytes are separated using the magnetic separation mechanism within 12 hours of harvesting of the first fluid sample. In some embodiments, the magnetic phagocytes are separated using the magnetic separation mechanism within 24 hours of harvesting of the first fluid sample. In some embodiments, the magnetic phagocytes are separated using the magnetic separation mechanism within 48 hours of harvesting of the first fluid sample. In some embodiments, the magnetic phagocytes are separated using the magnetic separation mechanism after the sample has been stored for a period of time.

As described above, in some embodiments, the methods herein may comprise subjecting the sample fluorescence-activated cell sorting (FACS). Fluorescence-activated cell sorting (FACS) is a type of flow cytometry that sorts a mixture of biological cells, one at a time, into separate containers based upon the specific light scattering and fluorescent characteristics of each cell. It provides quantitative recording of fluorescent signals from individual cells as well as physical separation of cells of particular interest. Generally, a current of a rapidly flowing stream of liquid carries a suspension of cells through a nozzle. The flow is selected such that there is a large separation between cells relative to their diameter. Vibrations at the tip of the nozzle cause the stream of cells to break into individual droplets, and the system is adjusted so that there is a low probability of more than one cell being in a droplet. A monochromatic laser beam illuminates the droplets, which are electronically monitored by fluorescent detectors. The droplets that emit the proper fluorescent wavelengths are electrically charged between deflection plates in order to be sorted into collection tubes.

The present invention is not limited to fluorescent assays, e.g., fluorescent microscopy or imaging. In some embodiments, the methods herein comprise colorimetric assays. As a non-limiting example, the methods may comprise a colorimetric ELISA. In some embodiments, the methods herein comprise imaging without a microscope. In some embodiments, the methods herein comprise using an image analysis system, wherein images may be obtained from surfaces such as a slide or a plate (e.g., microplate well), etc.

In some embodiments, the methods herein comprise sorting the phagocytes using a magnetic mechanism, e.g., magnetic extraction.

In some embodiments, the phagocytes are stained with a labeled phagocyte-specific binding moiety. In some embodiments, a target biomarker, e.g., a CNS-derived biomarker inside or on the surface of phagocytes) is stained with a different color (a second color) than the phagocyte-specific binding moiety (a first color). The methods may further comprise measuring a ratio of the first color to the second color, wherein the ratio of colors is indicative of an amount of target biomarker molecules inside or displayed on the cell surface of said phagocytes.

In some embodiments, the circulating phagocytes have a specific immunotype. In some embodiments, the circulating phagocytes are concentrated. In some embodiments, the circulating phagocytes are concentrated based on immunotype.

The methods herein may also comprise introducing a factor or combination of factors to the sample and/or the phagocytes and/or the fraction, wherein the fraction helps prevent apoptosis of the phagocytes. Non-limiting examples of factors that may be introduced includes epidermal growth factor (EGF), fetal bovine serum (FBS), other growth factors, a nutrient-rich medium, etc.

The present invention also features methods for preservation of samples for preserving the amount and/or structure and/or location of the CNS-derived biomarker(s) of interest (e.g., for preserving the amount and/or structure and/or location of the epitope(s) of interest). For example, the present invention provides methods for treating samples for the purposes of preserving the biomarker, e.g., via heat denaturation (wherein proteolytic enzymes or other factors are inhibited without affecting the biomarker, e.g., the epitope of the biomarker, to a large extent). Other methods of preservation may include freeze drying or other rapid freezing processes, application of heparin or other factors, modifying the pH of the sample, etc. The present invention is not limited to the aforementioned methods or compositions.

In some embodiments, the phagocytes obtained from the sample are permeabilized. In some embodiments, the phagocytes are lysed via various means, e.g., hypotonic solution treatment, detergent solution treatment, mechanical stress, etc.

### Biomarkers

Various neural-derived debris antigens or biomarkers (e.g., debris from brain tissue or other central nervous system tissue, antigens that would not normally be found outside of the brain, etc.) may be found inside or on the surface of recirculating phagocytes in the peripheral blood. Such debris antigens may be used to detect (or monitor) neurodegenerative or neuroinflammatory diseases (e.g., diseases as described herein). Debris from other diseases, such as relapse-remitting diseases, may be retrieved specifically from circulating phagocytes as well.

The biomarker (e.g., antigen, neurological condition-associated protein, etc.) may be present inside or on the surface of a circulating phagocyte, e.g., peripheral phagocyte. As used herein, the term “peripheral” refers to anything outside of brain tissue. For example, a peripheral phagocyte may be obtained from cerebrospinal fluid (CSF). Phagocytes may include monocytes, macrophages, and/or lymphocytes. Such circulating phagocytes may be found in tissues, cells, and/or fluids in the body, for example in blood, peripheral blood mononuclear cells (PBMCs), synovial fluid, cerebrospinal fluid (CSF), central nervous system tissues, cystic fluid, lymph fluid, ascites, pleural effusion, interstitial fluid, ocular fluids, vitreal fluid, urine the like, or a combination thereof. In some embodiments, the biomarker is an intracellular component. For example, the biomarker may be obtained from within a macrophage. In some embodiments, the macrophage sample is permeabilized. In some embodiments, the macrophage is lysed via various means, e.g., hypotonic solution treatment, detergent solution treatment, mechanical stress, etc. The fluid obtained for probing the phagocytes may be dependent on the disease or condition of interest.

In some embodiments, the biomarkers or antigens or neural-derived biomarkers (or neurodegenerative disease-associated proteins or biomarkers), or fragments thereof, include but are not limited to neuromelanin, a Tau protein (or fragment thereof), a Tau protein or fragment thereof comprising a phosphorylated residue (e.g., a phosphorylated serine reside, a phosphorylated threonine reside; e.g., serine 214, serine 235, serine 262, serine 356, serine 396, serine 404, serine 413, serine 46, serine 515, serine 516, serine 519, serine 531, serine 552, serine 610, serine 622, serine 641, serine 713, serine 721, serine 726, serine 730, serine 739, threonine 181, threonine 205, threonine 470, threonine 492, threonine 498, threonine 522, threonine 529, threonine 534, threonine 548); a protein or a fragment thereof selected from the group consisting of UCHL-1, neuromelanin or a fragment thereof, MBP, transketolase, NSI associated protein 1, major vault protein, synaptojanin, enolase, alpha synuclein, glial fibrillary acidic protein, S-100 protein, Neu-N, 26S proteasome subunit 9, ubiquitin activating enzyme ZE1, ubiquitin B precursor, vimentin, 13-3-3 protein, 14-3-3 protein (e.g., zeta isoform), NOGO-A, neuronal-specific protein gene product 9.5; proteolipid protein; myelin oligodendrocyte glycoprotein, GFAP, neurofilaments light chains (NFL), UCH-L1, amyloid beta, alpha-synuclein, apo A-I, Apo E, Apo J, JC viral antibody titers, TGF-beta, VEGF, dopamine-beta-hydroxylase (DBH), vitamin D binding protein, histidine-rich glycoprotein, cDNA FLJ78071, apolipoprotein C-II, immunoglobulin heavy constant gamma 3, alpha-1-acid glycoprotein 1, alpha-1-acid glycoprotein 2, haptoglobin-related protein, leucine-rich alpha-2-glycoprotein, or C-reactive protein, neuroglobin, valosin-containing protein, brain hexokinase, hippocalcin-1, nestin, synaptotagmin, myelin associated glycoprotein, myelin basic protein, myelin oligodendrocyte glycoprotein, myelin proteolipid protein, transketolase, NSI associated protein 1, major vault protein, synaptojanin, enolase, alpha synuclein, glial fibrillary acidic protein, S-100 proteinNeu-N, 26S proteasome subunit 9, annexin A2, annexin A3, annexin A5, annexin A6, annexin A11, ubiquitin activating enzyme ZE1, ubiquitin B precursor, vimentin, glyceraldehyde-3-phosphate dehydrogenase, 13-3-3 protein, 14-4-4 protein, neurofilament heavy chain and neurofilament light chain, TDP-43, superoxide dismutase (SOD), rhodopsin, all-spectrin breakdown products (SBDPs), tyrosinase EC 1.14.18.1, tyrosine hydroxylase, tyrosinase EC 1.14.16.2 (tyrosine 3-monooxygenase etc.), a synaptic antigen (e.g., PSD-95 protein, neurogranin, SNAP-25, TDP-43, etc.), etc. The present invention is not limited to the aforementioned biomarkers.

As a non-limiting example, neuromelanin may be detected in the debris of degenerated dopaminergic neurons in recirculating phagocytes. Abnormal neuromelanin levels may be associated with Parkinson's disease.

The term Tau biomarker may refer to full-length Tau, a fragment thereof, a particular epitope of Tau, e.g., an epitope within a particular region of amino acids. In some embodiments, the epitope is in a region from aa 240-441. In some embodiments, the epitope is in a region from aa 243-441. In some embodiments, the epitope is in a region from aa 244-274. In some embodiments, the epitope is in a region from aa 275-305. In some embodiments, the epitope is in a region from aa 306-336. In some embodiments, the epitope is in a region from aa 337-368. In some embodiments, the epitope is in a region from aa 388-411. The present invention is not limited to these regions. Further, the epitope may be in shorter regions of amino acids, e.g., aa 244-260, aa 270-280, aa 290-310, aa 330-360, etc. For example, the method may comprise detecting an epitope of Tau in the region of aa 201-441. In some embodiments, the method comprises detecting an epitope of Tau in the region from aa 243-441. In some embodiments, the method comprises detecting an epitope of Tau in the region from aa 244-274. In some embodiments, the method comprises detecting an epitope of Tau in the region from aa 275-305. In some embodiments, the method comprises detecting an epitope of Tau in the region from aa 306-336. In some embodiments, the method comprises detecting an epitope of Tau in the region from aa 337-368. In some embodiments, the method comprises detecting an epitope of Tau in the region from aa 388-411.

In some embodiments, phosphorylation of Tau can decrease its solubility. In some embodiments, the method comprises detecting a level of insoluble Tau protein in the sample. In some embodiments, an increased level of insoluble Tau protein as compared to a control level of insoluble Tau protein is indicative of the disease or condition of interest or a risk thereof.

In some embodiments, a combination (e.g., a pair) of biomarker-specific antibodies is used for isolating and detecting the biomarker of interest. The present invention includes a combination of any of the antibodies disclosed herein or antibodies specific to the biomarker of interest not necessarily listed herein, e.g., those that may be produced in the future, those that are commercially available, etc.

Without wishing to limit the present invention to any theory or mechanism, it is believed that in at least some cases, the biomarkers of interest are being cleaved or modified in a way that makes detection more difficult than isolating or detecting recombinant versions of the biomarker. For example, in some embodiments, a commercial antibody that is able to detect a recombinant biomarker may not work well to detect the biomarker. The present invention also features producing or identifying binding molecules (e.g., antibodies, fragments, etc.) that may be used to detect particular antigens or biomarkers of interest in the sample.

In some embodiments, more than one biomarker is detected in the sample(s). In some embodiments, the biomarker(s) is a neural-derived biomarker. However, the biomarker(s) is not limited to neural-derived biomarkers. In some embodiments, one or more biomarkers are detected in the sample, wherein the biomarkers are neural-derived, non-neural-derived biomarkers, or a combination thereof.

The biomarker may be of various lengths. For example, in some embodiments, the biomarker is from 5 to 20 amino acids. In some embodiments, the biomarker is from 20 to 40 amino acids. In some embodiments, the biomarker is from 40 to 80 amino acids. In some embodiments, the biomarker is from 80 to 150 amino acids. In some embodiments, the biomarker is from 150 to 200 amino acids. In some embodiments, the biomarker is from 200 to 300 amino acids. In some embodiments, the biomarker is from 300 to 400 amino acids. In some embodiments, the biomarker is from 400 to 500 amino acids. In some embodiments, the biomarker is from 500 to 600 amino acids.

The biomarker may comprise various regions of the full-length protein. For example, in some embodiments, the biomarker comprises the amino-terminus (e.g., N-terminus, NH2-terminus, N-terminal end, amino-terminus). The amino-terminus refers to the amino acid at the end of a protein or polypeptide that has a free amine group (—NH2). In some embodiments, the biomarker comprises about the first 15 amino acids. In some embodiments, the biomarker comprises about the first 25 amino acids. In some embodiments, the biomarker comprises about the first 50 amino acids. In some embodiments, the biomarker comprises about the first 75 amino acids. In some embodiments, the biomarker comprises about the first 100 amino acids. In some embodiments, the biomarker comprises about the first 125 amino acids.

In some embodiments, the biomarker or fragment thereof comprises the carboxy-terminus (e.g., C-terminus, COOH-terminus, C-terminal end, carboxyl-terminus). The carboxy-terminus refers to the amino acid at the end of a protein or polypeptide that has a free carboxylic acid group (—COOH). In some embodiments, the biomarker comprises the last 100 amino acids.

In some embodiments, the step of detecting the biomarker in the sample may comprise introducing an antibody to the sample, wherein the antibody binds to the biomarker. In some embodiments, the step of detecting the biomarker further comprises contacting the sample with a secondary antibody that binds to the primary antibody and detecting an antibody-biomarker complex. In some embodiments, detecting the antibody-biomarker complex indicates the presence of the particular disease or condition of investigation or a risk of the particular disease or condition of investigation.

In some embodiments, the sample is obtained from the subject, e.g., mammal, immediately following a relapse (e.g., exacerbation of symptoms) before a therapy (e.g., a steroid or other drug or other therapeutic intervention) treatment has begun. In some embodiments, the sample is obtained from the mammal before a relapse. In some embodiments, the sample is obtained during the course of the therapy treatment.

In some embodiments, the disease or interest associated with central nervous system tissue damage, central nervous system repair, neurodegeneration, etc., includes but is not limited to: autoimmune diseases, e.g., multiple sclerosis, rheumatoid arthritis, lupus, sjogren's syndrome, thyroiditis, uveitis, Crohn's disease, ulcerative colitis, psoriasis, type 1 diabetes mellitus, autoimmune Addison's disease, autoimmune hepatitis, celiac disease, pemphigous, chronic inflammatory demyelinating polyneuropathy, acute disseminated encephalomyelopathy, sarcoidosis, dermatomyositis and behcet's disease; neurological diseases or neurotrauma, e.g., stroke, concussion, chronic traumatic encephalopathy, neuromyelitis optica, transverse myelitis, intractable epilepsy and CNS infections; Parkinson's disease; primary tumor growth, metastasis of tumors; etc. Other diseases may include but are not limited to mild cognitive impairment, traumatic brain injury, Huntington's disease, amyotrophic lateral sclerosis, cerebral hypoxia, hydrocephalus, progressive supranuclear atrophy, corticobasal degeneration, multiple system atrophy, Lyme disease, and systemic lupus erythematosus, Neuromyelitis Optica, transverse myelitis, Acute and chronic Stroke, etc. The present invention is not limited to the aforementioned diseases. For example, the present invention may be used to detect a diabetes condition by detecting somatostatin in a similar manner as described herein, e.g., similar to methods for detecting neuromelanin for Parkinson's disease.

### Kits

The present invention also features kits for performing the methods described herein, e.g., detecting neural-derived biomarkers inside or on the surface of circulating phagocytes (from a sample derived from a subject, e.g., a mammal such as a human).

In some embodiments, the kit comprises one or more tools for processing samples (e.g., blood samples) and/or for collecting PBMCs, storing PBMCs, processing PBMCs, etc. In some embodiments, the kit comprises one or more tools for obtaining phagocytes from the PBMCs. In some embodiments, the kit comprises one or more tools for identifying phagocytes. In some embodiments, the kit comprises one or more tools for concentrating phagocytes.

The kit may comprise one or more binding moieties that bind to a specified appropriate biomarker and one or more binding moieties that bind to a phagocyte. In some embodiments, the kit further comprises a means for visualizing the binding of the binding moieties to the biomarker/antigen in the sample (e.g., an antibody-antigen complex), e.g., secondary antibodies.

In some embodiments, the binding moiety is a monoclonal or a polyclonal antibody. In some embodiments, the antibody is derived from a human, a mouse, a rat, a llama, a rabbit, a dog, a primate, a guinea pig, a cat, a hamster, a pig, a goat, a horse, or a cow. In some embodiments, the antibody is humanized. In some embodiments, the antibody is a chimera. In some embodiments, the binding moiety is an aptamer.

The antibodies in the kits may be purified, e.g., affinity purified for the antigen of interest. In some embodiments, the kits further comprise reagents for detecting additional biomarkers, e.g., additional biomarkers in the circulating phagocytes, serum biomarkers, plasma biomarkers, etc. In some embodiments, the kits further comprise reagents for capture and/or preserving the sample, e.g., for preserving the amount, structure, and/or location of the biomarker of interest (e.g., the epitope of the biomarker of interest). The kit may further comprise appropriate reagents, manuals, equipment, etc. For example, the kit may comprise slides coated with macrophage-specific binding moieties and reagents for automated assays. In some embodiments, the kit comprises reagents for multiplex assays.

### Applications of Analysis of Circulating Phagocytes

The present invention features methods of detecting and/or monitoring and/or treating diseases described herein, or detecting a risk thereof. For example, the present invention features methods for detecting and/or monitoring multiple sclerosis or detecting a risk of multiple sclerosis, methods of determining drug efficacy, methods for treating multiple sclerosis, etc. The present invention also features methods for detecting and/or monitoring Alzheimer's disease or detecting a risk of Alzheimer's disease, or methods for determining drug efficacy, etc. The present invention also features methods for treating Alzheimer's disease. The present invention also features methods for detecting and/or monitoring Parkinson's disease or detecting a risk of Parkinson's disease, or methods for determining drug efficacy, etc. The present invention also features methods for treating Parkinson's disease. The present invention also features methods for detecting and/or monitoring concussion or detecting a risk of concussion, methods of determining drug efficacy, etc. The present invention also features methods for treating concussion. The present invention also features methods for detecting and/or monitoring central nervous system tissue damage or detecting a risk of central nervous system tissue damage, or methods determining drug efficacy, etc. The present invention also features methods for treating central nervous system tissue damage. The present invention also features methods for detecting and/or monitoring central nervous system repair, methods for determining drug efficacy, etc.

The present invention also features methods for detecting and/or monitoring a relapse-remitting disease or detecting a risk of a relapse-remitting disease, methods for determining drug efficacy, etc. The present invention also features methods for treating a relapse-remitting disease. The relapse-remitting disease may be, for example, multiple sclerosis (MS), Lyme disease, rheumatoid arthritis (RA), systemic lupus erythematosus (SLE), Chron's disease, etc.

The present invention also features a method of detecting and/or monitoring an inflammatory condition in a similar manner, wherein the biomarker is associated with an inflammatory condition. The fluid obtained does not necessarily directly come into contact with the inflamed tissue being detected. For example, there may be a barrier between the fluid and the source of the biomarker. In other words, the fluid obtained may have once directly come into contact with the inflamed tissue, but at the time that it is being extracted in accordance with the present invention, it is being separated from the inflamed tissue by a barrier.

The biomarkers are detected in the recirculating phagocytes in the collected fluid sample. In some embodiments, two or more biomarkers are detected in a sample, e.g., a pattern of biomarkers may be detected in the sample. In some embodiments, the biomarker(s) is/are a neural-derived biomarker(s). However, the biomarker(s) is/are not limited to neural-derived biomarkers. In some embodiments, one or more biomarkers are detected in the sample, wherein the biomarkers are neural-derived, non-neural-derived biomarkers, or a combination thereof.

The method may feature comparing the amount of biomarker in the sample to a predetermined threshold or control for determining whether or not the level of biomarker in the sample is abnormal. As previously discussed, the predetermined threshold or control may be an industry standard (e.g., derived from non-disease controls) a laboratory standard, a patient standard, etc. For example the predetermined threshold or control may be, for example, a sample (a second sample) obtained from the patient at a time prior to obtaining the first sample.

In some embodiments, analyzing the level of the biomarker (e.g., an abnormal level of the biomarker) against the predetermined threshold or control level of the biomarker indicates the presence of the neurological condition, the disease, central nervous system tissue damage, CNS repair, etc., or a risk thereof.

Therapeutic compositions, treatments, drugs, or therapeutic interventions may include but are not limited to: crenezumab, mastinab, daclizumab, zinbryta, ER-Beta agonist, cyclophosphamide, rHIgM22, ponesimod, alpha-4 Integrin, AMP-110, an antisense oligonucleotide, Immune tolerizing agent, MultiStem, Kv1.3 Blocker, Alemtuzumab, IFNb-1b, BHT-3009-01, IFNb-1a, dimethyl fumarate, IFNb-1a (PEGylated), Natalizumab, MT1301, Abatacept, RCP1063, a compound related to Iamitrigene, CNM-Au8, Mesenchymal stem cell transplant, apolipoprotein E-based, modified peptidomimetic, Aimspro, Anti-BAFF Human Ab, NDC-1308, GNbAC1, Vatelizumab, Ofatumumab, ozanezumab (Nono-A mAb), Firategrast (SB-683699, T-0047), Interferon Alfa N3, Mitoxantrone, Ibudilast, Simvastatin, Raltegravir, Plovamer Acetate, Vedolizumab, Siponimod, Secukinumab, Fingolimod, Copaxone, LSD1-MAOB, Amiloride, adrenocorticotropic hormone, Rituximab, Ocrelizumab, Teriflunomide, glatiramer acetate, Laquinimod, TG-1101 (ublituximab), Clemastine Fumarate, Lipoic Acid, Minocycline, Riluzole, MIS416, Fibrin, ATX-MS1467, ARX424, Tcelna, TBC-4746, Feldetrex, PV-267, RNS60, R1295, VSN16R, Angiotensin AT2 agonist, GEH 120714, PDDIA, PDD3a, Zonisamide, deferiprone, NTCELL, Caffine, Isradipine, Rivastigmine, Inosine, Rasagiline, GM1 ganglioside, Nicotine, Exenatide, Pioglitazone, Glycerol Phenylbutyrate, EPI-743, an anti-inflammatory drug, ANX005, AADvac1, 5HT-6, Verubestat, PXT864, Donepezil, NPT088, RG1450, RG7412, PDE4E, PDE4B, Methylthioninium chloride, T3D-959, MultiTEP, ABT957, Anti-Tau antibodies, Aducanumab, Solanezumab, Inbrija, flurbiprofen, ketorolac, ketoprofen, tolmetin, aspirin, ibuprofen, naproxen, indomethacin, sulindac, piroxicam, mefenamic acid, meloxicam, diclofenac, celecoxib, etodolac, etoricoxib, lumiracoxib, rofecoxib, prodydlidine, diphenhydramine, benztropine, trihexyphenidyl, biperidin, bromocriptine, safinamide, selegiline, rotigotine, pramipexole, apomorphine, rasagiline, ropinirole, entacapone, amantadine, pergolide, carbidopa, levodopa, tolcapone, metformin, alogliptin, canagliflozin, dapagliflozin, empagliflozin, glipizide, or any other appropriate drug or combination of drugs or other therapeutic interventions. The present invention is not limited to the aforementioned therapeutic compositions or therapeutic interventions.

The present invention also features methods of selecting a patient for a clinical trial for testing a drug for an effect on central nervous system (CNS) tissue damage, CNS repair, or neurodegeneration. The method comprises detecting a level of a biomarker in the phagocytes, the biomarker being associated with CNS tissue damage, CNS system repair, or neurodegeneration, wherein if the level of the biomarker in the phagocytes is abnormal, then the patient has active central nervous system disease. The method may comprise selecting the patient for the clinical trial if the patient has active CNS disease. Without wishing to limit the present invention to any theory or mechanism, it is believed that the cohorts of patients for clinical trials created using the methods herein will be better suited to test a particular therapeutic intervention or composition of interest. Further, these cohorts may be used to reevaluate drugs that may have previously failed in clinical trials (e.g., crenezumab), because the drugs may have failed due to the cohort of patients not being appropriate for the testing. Patients exhibiting low current disease activity may be poor responders or non-responder to therapeutic treatments.

In some embodiments, the biomarker is one that helps define subgroups of a particular disease state or disease process. In some embodiments, the biomarker is TDP-43, which may be used to help define subgroups of ALS.

Table 1 shows the relationship between several biomarkers, such as Tau, GFAP, NFL, UCH-L1, etc., and the active disease processes associated with diseases such as Multiple Sclerosis, Alzheimer's disease, Parkinson's disease, etc. Referring to Table 1, ↑=elevated compared to controls, ↓=decreased compared to controls, −=no change compared to controls, Eq.=equivocal, C=cerebrospinal fluid, S=serum, P=plasma, B=blood (plasmas=/serum not specified), E=exosomes, R=recirculating phagocytes. Additional biomarkers: ¹MOG: ↑^{c}, ¹S100B: ↑^{c}, ²P-tau: ↑^{E}, ³P-tau: −^{c}.

TABLE 1

| | | | | | Amyloid | Alpha- | Neuro |
|---|---|---|---|---|---|---|---|
| | Tau | GFAP | NFL | UCH-L1 | Beta | Synuclein | melanin |
| Multiple | ↑^{C,S,R} | ↑^{C,S} | ↑^{C,S} | ↑^{R} | ↓^{C} | ↓^{C} | ↑^{R} |
| Sclerosis¹ | | | | | | | |
| Alzheimer's | ↓^{C,P,R} ,__E | ↑^{C} | ↑^{C,S,P} | ↑^{C,B,R} | ↓^{C}, ↑^{E} | ↑^{C} | ↑^{R} |
| Disease² | | | | | | | |
| Mild Cognitive | | | ↑^{C,S,P} | | | ↑^{C} | |
| Impairment | | | | | | | |
| Parkinson's | ↓^{C}, __P, | __C, ↑^{S} | __C,B | ↓^{C}, ↑^{R} | __C | ↓^{C}, Eq.^{P} , ↑^{P} | ↑^{R} |
| Disease | ↑^{E,R} | | | | | | |
| Multiple System | Eq. | __C | ↑^{C,B} | ↓^{C} | __C | ↓^{C}, ↑^{P} | |
| Atrophy | | | | | | | |
| Progressive | | __C | ↑^{C,B} | ↓^{C} | | __C | |
| Supranuclear | | | | | | | |
| Palsy | | | | | | | |
| Corticobasal | | __C | ↑^{C,B} | | | __C | |
| Degeneration | | | | | | | |
| Amyotrophic | ↑^{C} | | ↑^{C,S,P} | | | | |
| Lateral | | | | | | | |
| Sclerosis³ | | | | | | | |
| Huntington's | | | ↑^{C,P} | | | | |
| Disease | | | | | | | |
| Traumatic Brain | ↑^{C,P,R} | ↑^{C,S,P} | ↑^{C,S} | ↑^{C,S,P,R} | __C, ↑^{P} | | ↑^{R} |
| Injury | | | | | | | |

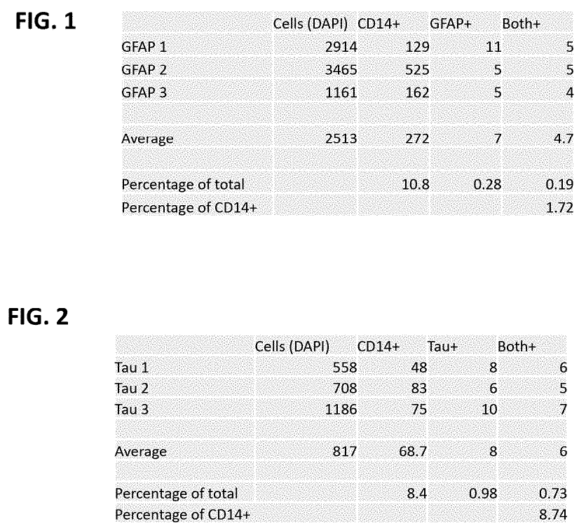

FIG. 1 shows the results of GFAP fluorescent imaging and cell counts. Three image planes (GFAP1, GFAP2, GFAP3) from one sample were imaged at 5× and fluorescent cells (DAPI[350]/CD14[568]/GFAP[488]) were counted using ImageJ software. Cell counts were fairly high (low thousands). This sample contained on average 10.8% CD14+ cells and of those cells roughly 1.7% were GFAP+(0.19% of total). There were a few cells which were GFAP+/CD14−.

FIG. 2 shows Tau the results of fluorescent imaging and cell counts. Three image planes (Taut, Tau2, Tau3) from one sample were imaged at 5× and fluorescent cells (DAPI[350]/CD14[568]/TAU[488]) were counted using ImageJ software. Cell counts were somewhat lower (due to division of sample between several slides for troubleshooting). This sample contained on average 8.4% CD14+ cells and of those cells roughly 8.7% were TAU+(0.7% of total). There were a few cells which were TAU+/CD14−.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and for ease of review by the patent office only and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

What is claimed is:

1. A method, comprising:
a. producing a preparation comprising CNS-derived molecules by introducing to a whole blood sample obtained from outside central nervous system (CNS) tissue of a subject a first detectable binding moiety specific for circulating neutrophils and a second detectable binding moiety specific for a CNS-derived molecule, the first detectable binding moiety being differentially detectable from the second detectable binding moiety;
b. subjecting the preparation to single-cell analysis for detecting the first detectable binding moiety and second detectable binding moiety; and
c. analyzing the CNS-derived molecules in the preparation.

2. The method of claim 1, wherein the single-cell analysis is flow cytometry.

3. The method of claim 1, wherein the single-cell analysis is a single cell enzyme linked immunosorbent assay (ELISA).

4. The method of claim 1, wherein the single-cell analysis is a microscopy-based assay.

5. The method of claim 1, wherein single-cell analysis comprises placing the preparation on a solid surface, using said surface as a wave guide for illumination, and imaging by direct charge-coupled device (CCD).

6. The method of claim 1, wherein the first detectable binding moiety, the second detectable binding moiety, or both comprise a fluorescent label, a fluorescent antibody, a nanoparticle, a quantum dot, or a tag.

7. The method of claim 1, wherein the CNS-derived molecule comprises one or a combination of: Tau, phosphorylated Tau, hippocalcin-1, 14-3-3 protein, MBP, UCH-L1, TDP-43, superoxide dismutase (SOD), neuromelanin, glial fibrillary acidic protein (GFAP), neurofilament light chain (NFL), neurofilament heavy chain (NFH), neurofilament medium chain (NFM), phosphorylated NFL, phosphorylated NFH, phosphorylated NFM, internexin (Int), peripherin, UCH-L1, amyloid beta, alpha-synuclein, apo A-I, Apo E, Apo J, a viral antigen, a JC viral antigen, TGF-beta, VEGF, dopamine-beta-hydroxylase (DBH), vitamin D binding protein, histidine-rich glycoprotein, cDNA FLJ78071, apolipoprotein C-II, immunoglobulin heavy constant gamma 3, alpha-1-acid glycoprotein 1, alpha-1-acid glycoprotein 2, haptoglobin-related protein, leucine-rich alpha-2-glycoprotein, erythropoietin (EPO), C-reactive protein, tyrosinase EC 1.14.18.1, tyrosine hydroxylase, tyrosinase EC 1.14.16.2, PSD-95 protein, neurogranin, SNAP-25, TDP-43, transketolase, NSI associated protein 1, major vault protein, synaptojanin, enolase, alpha synuclein, S-100 protein, Neu-N, 26S proteasome subunit 9, ubiquitin activating enzyme ZE1, ubiquitin B precursor, vimentin, 13-3-3 protein, NOGO-A, neuronal-specific protein gene product 9.5, proteolipid protein; myelin oligodendrocyte glycoprotein, neuroglobin, valosin-containing protein, brain hexokinase, nestin, synaptotagmin, myelin associated glycoprotein, myelin basic protein, myelin oligodendrocyte glycoprotein, myelin proteolipid protein, annexin A2, annexin A3, annexin A5, annexin A6, annexin A11, ubiquitin activating enzyme ZE1, ubiquitin B precursor, vimentin, glyceraldehyde-3-phosphate dehydrogenase, 14-4-4 protein, rhodopsin, all-spectrin breakdown products (SBDPs), or a breakdown product thereof.

8. A method for measuring a biomarker inside of or displayed on a cell surface of neutrophils in a sample from a subject, said method comprising:
a. staining neutrophils in the sample from the subject with a labeled first binding moiety specific for neutrophils, and staining at least one target biomarker molecule with a labeled second binding moiety specific for the target biomarker, the first binding moiety can be differentiated from the second binding moiety, the target biomarker being a compound derived from central nervous system tissue that is within or displayed on the cell surface of the neutrophils; and
b. detecting the first binding moiety and the second binding moiety, wherein the relationship between the amount or distribution of the first binding moiety and second binding moiety is indicative of an amount of target biomarker molecules inside or displayed on the cell surface of said neutrophils.

9. The method of claim 1, wherein the sample is whole blood.

10. The method of claim 8, wherein detecting the binding moieties comprises subjecting the sample to flow cytometry or ELISA.

11. The method of claim 8, wherein the neutrophils are immobilized prior to staining.

12. The method of claim 8, wherein the labels of the binding moieties are fluorescent labels.

13. The method of claim 8, wherein the target biomarker molecule is associated with neurologic disease or neurotrauma.

14. The method of claim 8, wherein the target biomarker is one or a combination of: Tau, phosphorylated Tau, hippocalcin-1, 14-3-3 protein, MBP, UCH-L1, TDP-43, superoxide dismutase (SOD), neuromelanin, glial fibrillary acidic protein (GFAP), neurofilament light chain (NFL), neurofilament heavy chain (NFH), neurofilament medium chain (NFM), phosphorylated NFL, phosphorylated NFH, phosphorylated NFM, internexin (Int), peripherin, UCH-L1, amyloid beta, alpha-synuclein, apo A-I, Apo E, Apo J, a viral antigen, a JC viral antigen, TGF-beta, VEGF, dopamine-beta-hydroxylase (DBH), vitamin D binding protein, histidine-rich glycoprotein, cDNA FLJ78071, apolipoprotein C-II, immunoglobulin heavy constant gamma 3, alpha-1-acid glycoprotein 1, alpha-1-acid glycoprotein 2, haptoglobin-related protein, leucine-rich alpha-2-glycoprotein, erythropoietin (EPO), C-reactive protein, tyrosinase EC 1.14.18.1, tyrosine hydroxylase, tyrosinase EC 1.14.16.2, PSD-95 protein, neurogranin, SNAP-25, TDP-43, transketolase, NSI associated protein 1, major vault protein, synaptojanin, enolase, alpha synuclein, S-100 protein, Neu-N, 26S proteasome subunit 9, ubiquitin activating enzyme ZE1, ubiquitin B precursor, vimentin, 13-3-3 protein, NOGO-A, neuronal-specific protein gene product 9.5, proteolipid protein; myelin oligodendrocyte glycoprotein, neuroglobin, valosin-containing protein, brain hexokinase, nestin, synaptotagmin, myelin associated glycoprotein, myelin basic protein, myelin oligodendrocyte glycoprotein, myelin proteolipid protein, annexin A2, annexin A3, annexin A5, annexin A6, annexin A11, ubiquitin activating enzyme ZE1, ubiquitin B precursor, vimentin, glyceraldehyde-3-phosphate dehydrogenase, 14-4-4 protein, rhodopsin, all-spectrin breakdown products (SBDPs), or a breakdown product thereof.
